(19) United States
(12) Patent Application Publication
(10) Pub. No.: US 2021/0137861 A1
(43) Pub. Date: May 13, 2021

(54) METHODS FOR REDUCING ACCUMULATED PATHOLOGIC TAU PROTEIN

(71) Applicant: HOUGH EAR INSTITUTE, OKLAHOMA CITY, OK (US)

(72) Inventor: Richard D. KOPKE, Oklahoma City, OK (US)

(73) Assignee: HOUGH EAR INSTITUTE, OKLAHOMA CITY, OK (US)

(21) Appl. No.: 17/052,093

(22) PCT Filed: May 1, 2019
(86) PCT No.: PCT/US2019/030184

§ 371 Date: Oct. 30, 2020

Related U.S. Application Data
(60) Provisional application No. 62/666,459, filed on May 3, 2018.

(51) Int. Cl. A61K 31/185; A61K 31/198; A61K 45/06; A61P 27/16; A61P 25/28

(52) U.S. Cl. CPC A61K 31/185; A61K 31/198; A61P 25/28; A61P 27/16; A61K 45/06

(57) ABSTRACT

Disclosed here is a method for reducing accumulated pathologic Tau proteins in a subject in need thereof, comprising administering to the subject an effective amount of 2,4-disulfonyl α-phenyl tertiary butyl nitrone (2,4-DSPBN) or a pharmaceutically acceptable salt thereof. The method may further comprise administrating N-acetylcysteine (NAC) to the subject in combination with 2,4-DSPBN.

### BACKGROUND

Blast-induced and noise-induced accumulation of hyperphosphorylated and oligomeric Tau can occur in the hippocampus. Aberrant hyperphosphorylation and oligomerization of this microtubule-associated protein can initiate progressive pathological processes that culminate in synaptic loss and neuronal cell death. Based on the fundamental etiological role that Tau dysfunction plays in Alzheimer's and related diseases (tauopathies), much emphasis has been placed on identifying therapeutic strategies to block or delay the propagative chain of events that lead to Tau-induced neurotoxicity. It is unclear, however, whether and how preexisting accumulation or aggregation of pathologic Tau can be reversed in patients suffering from chronic neurodegeneration disorders and/or chronic otologic diseases, including those affecting the peripheral auditory system.

Therefore, a need exists for methods for reversing pathologic Tau protein accumulation or aggregation associated with chronic neurodegeneration disorders and/or chronic otologic diseases.

### SUMMARY

The present invention met the aforementioned need by providing methods and compositions for reversing accumulation of pathologic Tau proteins in patients suffering from chronic diseases or conditions associated with accumulated pathologic Tau proteins. Accordingly, at least one aspect of the invention described herein relates to a method for reducing accumulated pathologic Tau proteins in a subject, comprising administering to said subject in need thereof an effective amount of 2,4-disulfonyl α-phenyl tertiary butyl nitrone (2,4-DSPBN) or a pharmaceutically acceptable salt thereof.

In some embodiments, the 2,4-DSPBN or pharmaceutically acceptable salt thereof is administered as a pharmaceutical composition, which further comprises a pharmaceutically acceptable carrier.

In some embodiments, the 2,4-DSPBN or pharmaceutically acceptable salt thereof is administered to the subject orally, intravenously, subcutaneously, sublingually, subdermally, intrathecally, by inhalation, or locally within an ear.

In some embodiments, the method further comprises administering one or more compounds selected from the group consisting of N-acetylcysteine, Acetyl-L-Carnitine, glutathione monoethylester, ebselen, D-methionine, carbamathione and Szeto-Schiller peptides and their functional analogs. In some embodiments, the method further comprises administering N-acetylcysteine.

In some embodiments, the accumulated pathologic Tau protein is caused by aging.

In some embodiments, the accumulated pathologic Tau protein is caused by a central nervous system disease. In some embodiments, the 2,4-DSPBN or pharmaceutically acceptable salt thereof is administered to the subject at least two months, at least three months, at least six months, at least nine months, at least twelve months, or 1-60 months after onset of the central nervous system disease.

In some embodiments, the accumulated pathologic Tau protein is caused by exposure to noise. In some embodiments, the 2,4-DSPBN or pharmaceutically acceptable salt thereof is administered to the subject at least two months, at least three months, at least six months, at least nine months, at least twelve months, or 1-60 months after the exposure to noise.

In some embodiments, the accumulated pathologic Tau is caused by exposure to blast. In some embodiments, the 2,4-DSPBN or pharmaceutically acceptable salt thereof is administered to the subject at least two months, at least three months, at least six months, at least nine months, at least twelve months, or 1-60 months after the exposure to blast.

In some embodiments, the accumulated pathologic Tau is caused by infection. In some embodiments, the 2,4-DSPBN or pharmaceutically acceptable salt thereof is administered to the subject at least two months, at least three months, at least six months, at least nine months, at least twelve months, or 1-60 months after the infection.

In some embodiments, the accumulated pathologic Tau is caused by exposure to toxin. In some embodiments, the 2,4-DSPBN or pharmaceutically acceptable salt thereof is administered to the subject at least two months, at least three months, at least six months, at least nine months, at least twelve months, or 1-60 months after the exposure to toxin.

In some embodiments, the subject suffers from chronic noise-induced or blast-induced hearing loss or tinnitus, or Presbycusis or Presbycusis-associated tinnitus, and the administration of the 2,4-DSPBN or pharmaceutically acceptable salt thereof reduces the accumulated pathologic Tau protein in the cochlear or vestibular region by at least 1%, or at least 2%, or at least 5%, or at least 10%, or at least 20%, or at least 50%.

In some embodiments, the subject suffers from a central nervous system disease, and the administration of the 2,4-DSPBN or pharmaceutically acceptable salt thereof reduces the accumulated pathologic Tau protein in the central nervous system by at least 1%, or at least 2%, or at least 5%, or at least 10%, or at least 20%, or at least 50%.

In some embodiments, cerebrospinal fluid of the subject comprises at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein.

In some embodiments, the subject suffers from a central nervous system disease or condition selected from chronic traumatic encephalopathy, Alzheimer's disease, Parkinson's disease, progressive supranuclear palsy, frontotemporal dementia, Pick's disease, Argyrophilic grain dementia, corticobasal degeneration, progressive subcortical gliosis, amyotrophic lateral sclerosis, diffuse neurofibrillary tangles with calcification, dementia pugilistica, tangle-only dementia, Down's syndrome, Gerstmann-Straussler-Scheinker disease, Hallervorden-Spatz disease, Creutzfeldt-Jakob disease, multiple system atrophy, Niemann-Pick disease type C, prion protein cerebral amyloid angiopathy, subacute sclerosing panencephalitis, myotonic dystrophy, non-Guanamian motor neuron disease with neurofibrillary tangles, and postencephalitic parkinsonism.

In another aspect, the invention described herein relates to a composition for use in a method for reducing accumulated pathologic Tau proteins in a subject, comprising a pharmaceutically effective amount of a composition comprising 2,4-disulfonyl α-phenyl tertiary butyl nitrone (2,4-DSPBN) or a pharmaceutically acceptable salt thereof and N-acetylcysteine (NAC) or a pharmaceutically acceptable salt thereof.

In another aspect, the invention described herein relates to a composition for use in a method for reducing accumulated pathologic Tau proteins in a subject, comprising a pharmaceutically effective amount of a composition comprising 2,4-disulfonyl α-phenyl tertiary butyl nitrone (2,4-DSPBN) or a pharmaceutically acceptable salt thereof.

These and other features, together with the organization and manner of operation thereof, will become apparent from the following detailed description when taken in conjunction with the accompanying drawings.

### BRIEF DESCRIPTION OF THE DRAWINGS

FIG. 1 shows T22 positive neuron counting and statistical analyses in the spiral ganglion (SG) after noise exposure. Rats were exposed to 115 dB SPL octave band noise for one hour. One group of rats were treated with 2,4-DSPBN and NAC (300 mg/kg of each) one hour after noise exposure and twice/day for the next two days (total 5 doses, the N/T group) while another group of rats were treated with vehicle (saline) only (the N group). One group of rats without noise exposure served as normal controls (the NC group). Animals were euthanized at 7- and 21-days after noise exposure, and cochlear tissues were processed for immunostaining. An increased number of T22-positive neurons were observed in the SG at 7 days after noise exposure in the 7D-N/T and 7D-N groups compared to the NC group (**, *** p<0.01 or 0.001). No treatment effect was observed at this time point after noise exposure (p>0.05). At twenty-one days after noise exposure, an increased number of T22-positive neurons was observed in the SG only in the vehicle-treated group (21D-N, *** p<0.001) but not in the group treated with a combination of 2,4-DSPBN+NAC (21D-N/T, p>0.05), indicating a treatment effect at this time point (p<0.05).

FIG. 2 shows that 2,4-DSPBN+NAC treatment significantly reduced the accumulation of insoluble cytotoxic Tau aggregates in the brains of Tau(P301S) transgenic mice. Transgenic (Tg) Tau(P301S) mice are an aggressive tauopathy model that begins demonstrating neurodegeneration and accumulation of hyperphosphorylated Tau oligomers within the first five months of age. Mice were treated (i.p.) daily for three months with either placebo (saline) or 2,4-DSPBN+NAC (300 mg/kg of each), beginning at an age of 2.5 months. Differential fractionation of brain tissues and immunoblot analyses were conducted among the Tg mice at 5.5 months to measure the relative accumulation of detergent (sarkosyl)-insoluble aggregates of hyperphosphorylated Tau protein in animals treated with the placebo or the active drug combination, using methodology described by Berger et al., J Neurosci. 27(14):3650-62 (2007). FIG. 2A depicts immunoblot analyses conducted among the sarkosyl-insoluble fractions isolated from hippocampal (HCa) brain tissues from Tau(P301S)-Tg mice treated with either placebo or 2,4-DSPBN+NAC. In FIG. 2A, the relative amounts of aggregated hyperphosphorylated Tau (p-Tau (S202, T205)) that remained in the sarkosyl-insoluble pellet of the HCa extracts were detected, using an antibody (AT8) specific to p-Tau (S202,T205). The levels of aggregated insoluble p-Tau(S202, T205) in P301S-Tg mice treated with 2,4-DSPBN+NAC were noticeably reduced relative to placebo-treated controls. FIG. 2B is a graphical summary depicting quantitative comparisons of densitometric immunoreactivity of p-Tau in the sarkosyl-insoluble fraction of the HCa from mice in each test group (n=6 mice/group). These data showed a statistically-significant reduction in the amount of cytotoxic sarkosyl-insoluble p-Tau aggregation in Tg mice treated with 2,4-DSPBN+NAC. Immunoblots of the housekeeping protein, GAPDH, in the soluble supernatant fractions from the same samples served as internal loading controls for quantitative comparisons.

FIG. 3 shows that 2,4-DSPBN+NAC treatment reduced okadaic acid-induced p-Tau accumulation in SH-SY5Y neuroblastoma cells. Reproducible hyperphosphorylated p-Tau accumulation can be induced in the SH-SY5Y neuroblastoma cell line following incubation with Okadaic Acid (OA), a small molecule inhibitor of protein phosphatase 2A (PP2A), according to the method described by Boban et al., J Neurosci Methods., S0165-0270(18)30297-8 (2018). In this experiment, SH-SY5Y cells were incubated with 100 nM OA alone or in combination with 2,4-DSPBN+NAC for three hours prior to harvesting the cells for immunoblot evaluations with the AT8 phospho-Tau (S202,T205) antibody or the housekeeping protein, GAPDH. In cells treated with OA alone, marked phospho-Tau(S202,T205) accumulation was observed relative to untreated control cells (compare lanes 1 and 2). Cells treated with OA and 2,4-DSPBN+NAC, showed markedly reduced accumulation of p-Tau levels relative to cells treated with OA alone (compare lanes 2 and 4 or lanes 2 and 5). In the absence of OA, 2,4-DSPBN+NAC treatment (500 μM NAC, 185 μM 2,4-DSPBN) alone also seemingly reduced the homeostatic baseline levels of p-Tau in SH-SY5Y cells (compare lanes 1 and 3). Two doses of combinatorial 2,4-DSPBN+NAC (185 μM 2,4-DSPBN/500 μM NAC or 93 μM 2,4-DSPBN/250 μM NAC) were used in this experiment, each of which used a molar ratio that mimics the equal mass:mass ratios used in the in vivo studies (i.e. a 1:1 mass ratio of 2,4-DSPBN and NAC is equivalent to a 1:2.7 molar ratio).

FIG. 4 shows that quantitative evaluations demonstrated a statistically-significant reduction in okadaic acid-induced p-Tau accumulation in SH-SY5Y neuroblastoma cells treated with 2,4-DSPBN+NAC. Biological and technical replicates of the experiment described in FIG. 3 were conducted, and p-Tau(S202,T205) immunoblots (AT8 antibody) were subjected to densitometric evaluations of relative protein levels, using National Institutes of Health ImageJ software and the internal GAPDH protein standard. Based on these evaluations, it was demonstrated that 2,4-DSPBN+NAC treatment (500 μM NAC, 185 μM 2,4-DSPBN) promoted a statistically-significant reduction in OA-induced p-Tau accumulation, to a level that was statistically-indistinguishable from the levels observed in untreated control cells, indicative of a strong treatment effect.

FIG. 5 shows that 2,4-DSPBN+NAC treatment enhanced the reversal of accumulated phosphorylated Tau protein levels. In this pilot experiment, SH-SY5Y cells were challenged with OA alone for 3 hours and then chased with fresh media without OA but containing either saline or 2,4-DSPBN+NAC (500 μM NAC, 185 μM 2,4-DSPBN). Cells were then harvested at 30 min. post-treatment to examine whether 2,4-DSPBN+NAC treatment might accelerate the resolution of the accumulated p-Tau induced by OA. FIG. 5A shows an immunoblot evaluation of hyperphosphorylated p-Tau(S202,T205) levels in SH-SY5Y cells, which depicts a persistent marked OA-induced accumulation that was still evident after 30 min in OA-free media, whereas cells treated with 2,4-DSPBN+NAC showed a detectable reduction at this same timepoint post-OA. FIG. 5B shows quantitative densitometric evaluations of technical replicates of this experiment, demonstrating that treatment with 2,4-DSPBN+NAC seemed to more rapidly resolve the OA-induced accumulation of p-Tau in SH-SY5Y cells.

### DETAILED DESCRIPTION

The invention described herein provides a method for reducing accumulated pathologic Tau proteins (i.e., reversing Tau accumulation) in a subject, comprising administering to said subject in need thereof an effective amount of 2,4-disulfonyl α-phenyl tertiary butyl nitrone (2,4-DSPBN, or HPN-07) or a pharmaceutically acceptable salt thereof. Optionally, the 2,4-DSPBN is co-administered with NAC.

Abbreviations

AGD, argyrophilic grain dementia; ALS, amyotrophic lateral sclerosis; CBD, corticobasal degeneration; CJD, Creutzfeldt-Jakob disease; CTE, chronic traumatic encephalopathy; DNTC, diffuse neurofibrillary tangles with calcification; DP, dementia pugilistica, 2,4-DSPBN, 2,4-disulfonyl α-phenyl tertiary butyl nitrone; FTD, frontotemporal dementia; GAPDH, glyceraldehyde 3-phosphate dehydrogenase; GSS, Gerstmann-Straussler-Scheinker disease; HCa, hippocampus; HSD, Hallervorden-Spatz disease; i.p., intraperitoneal; MSA, multiple system atrophy; NAC, N-acetylcysteine; NPC, Niemann-Pick disease type C; OA, okadaic acid; 4-OHPBN, 4-hydroxy-α-phenyl butyl nitrone; PBN, phenyl butyl nitrone ; PEP, postencephalitic parkinsonism; PrP-CAA, prion protein cerebral amyloid angiopathy; PSP, progressive supranuclear palsy; SG, spiral ganglion; S-PBN, 2-sulfonyl-α-phenyl tertiary butyl nitrone; SSPE, subacute sclerosing panencephalitis; Tg, transgenic; TOD, tangle-only dementia.

2,4-disulfonyl α-phenyl tertiary butyl nitrone (2,4-DSPBN)

2,4-disulfonyl α-phenyl tertiary butyl nitrone is also referred to as 2,4-disulfonyl PBN, 2,4-DSPBN, NXY-059, HPN-07, or CAS 168021-79-2. It has the following structure:

[chemical structure]

The acid form of the compound has the following structure:

[chemical structure]

The acid form may be a solid or found in low pH solutions. The ionized salt form of the compound exists at higher pH and may be represented by either of the following structures:

[chemical structure]

In the salt form, X is a pharmaceutically acceptable cation. Most commonly, this cation is a monovalent material such as sodium, potassium or ammonium, but it can also be a multivalent alone or cation in combination with a pharmaceutically acceptable monovalent anion, for example calcium with a chloride, bromide, iodide, hydroxyl, nitrate, sulfonate, acetate, tartrate, oxalate, succinate, pamoate or the like anion; magnesium with such anions; zinc with such anions or the like. Among these materials, the free acid and the simple sodium, potassium or ammonium salts are most preferred with the calcium and magnesium salts also being preferred but somewhat less so. 2,4-DSPBN is described in detail by U.S. Pat. No. 5,488,145, which is incorporated herein by reference. The salts of 2,4-DSPBN may also be used for reducing accumulated pathologic Tau protein in a manner similar to the use of 2,4-DSPBN as described herein.

To reduce accumulated pathologic Tau protein, 2,4-DSPBN can be administered at a dose of, for example, between about 1 mg/kg to about 500 mg/kg body weight, or between about 5 mg/kg to about 400 mg/kg body weight, or between about 10 mg/kg to about 300 mg/kg body weight, or at about 10 mg/kg body weight, or at about 20 mg/kg body weight, or at about 50 mg/kg body weight, or at about 100 mg/kg body weight, or at about 150 mg/kg body weight, or at about 200 mg/kg body weight, or at about 250 mg/kg body weight, or at about 300 mg/kg body weight.

To reduce accumulated pathologic Tau protein in a human subject, 2,4-DSPBN can be administered at a daily dose of, for example, between about 100 mg to about 20,000 mg, or between about 500 mg to about 10,000 mg, or between about 1,000 mg to about 5,000 mg, or at about 100 mg, or at about 200 mg, or at about 500 mg, or at about 1,000 mg, or at about 2,000 mg, or at about 3,000 mg, or at about 5,000 mg, or at about 8,000 mg, or at about 10,000 mg.

The subject can be administered one dose daily, or two doses daily, or three doses daily, or four doses daily, or five doses daily.

2,4-DSPBN can be combined with NAC to reduce accumulated pathologic Tau protein. In some embodiments, 2,4-DSPBN and NAC are co-administered as a mixture. In some embodiments, 2,4-DSPBN and NAC are administered sequentially or simultaneously as distinct dosage forms.

In some embodiments, 2,4-DSPBN, and optionally NAC, are administered orally. Other delivery methods including, but not limited to, intravenously, subcutaneously, by inhalation, sublingually, subdermally, intrathecally, or locally within the ear are envisioned. Further, the active composition may be administered as a nanoparticle or dendrimer formulation. The nanoparticle may be multifunctional and composed of a polymer and paramagnetic iron oxide particles to allow the application of external magnetic forces to aid in the delivery of the drug to the desired target such as the inner ear or the dorsal cochlear nucleus. Additionally, the composition may be formulated with additives known to those skilled in the art to enhance oral absorption and alter bioavailability kinetics.

In place of or in addition to 2,4-DSPBN, other nitrone compounds can also be used to reduce accumulated pathologic Tau protein. In some embodiments, the nitrone compound is selected from phenyl butyl nitrone (PBN) and its derivatives. In some embodiments, the nitrone compound is PBN. In some embodiments, the nitrone compound is 4-hydroxy-α-phenyl butyl nitrone (4-OHPBN). In some embodiments, the nitrone compound is 2-sulfonyl-α-phenyl tertiary butyl nitrone (S-PBN).

Therefore, the present application expressly covers the use of any of the aforementioned nitrone compounds in place of or in addition to 2,4-DSPBN in all embodiments disclosed herein. Hence, methods are disclosed in which one or more of phenyl butyl nitrone (PBN), 4-hydroxy-α-phenyl butyl nitrone (4-OHPBN) and 2-sulfonyl-α-phenyl tertiary butyl nitrone (S-PBN) are used in place of or in addition to the 2,4-DSPBN.

Methods of Reducing Accumulated Pathologic Tau Proteins

Many embodiments described herein relate to a method for reducing accumulated pathologic Tau proteins in a subject, comprising administering to said subject in need thereof an effective amount of 2,4-disulfonyl α-phenyl tertiary butyl nitrone (2,4-DSPBN) or a pharmaceutically acceptable salt thereof. Optionally, the 2,4-DSPBN is co-administered with NAC. In another embodiment, 2,4-DSPBN is co-administered with a Tau aggregation inhibitor.

In this disclosure, the terms “Tau” refers to the native monomer form of Tau, or other conformers of Tau, for example, oligomers or aggregates of Tau. The term “Tau” is also used to refer collectively to all types and forms of Tau. In some forms, the aggregates of Tau include hyperphosphorylated Tau proteins. In some embodiments, hyperphosphorylated Tau may be detected by using any antibody known to be immunoreactive to the hyperphosphorylated form of Tau in combination with immunoassays well-known in the art. In some embodiments, hyperphosphorylated Tau can be, for instance, detected with an antibody (AT8) specific to p-Tau phosphorylated at positions 202 and 205 (S202, T205). In some embodiments, hyperphosphorylated Tau can be, for instance, detected with the PHF-1 (high specificity for the S396, S404 phospho-epitope), AT180, and AT270 antibodies known in the art to recognize hyperphosphorylated Tau. Tau proteins perform the function of stabilizing microtubules, which are abundant in nerve cells and are present to a much lesser degree in oligodendrocytes and astrocytes. When Tau proteins become defective and fail to adequately stabilize microtubules, pathologies of the nervous system can develop such as Alzheimer's disease. In some embodiments, cerebrospinal fluid of the subject comprises at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein.

In one embodiment, the 2,4-DSPBN or pharmaceutically acceptable salt thereof is administered as a pharmaceutical composition, which further comprises a pharmaceutically acceptable carrier. In another embodiment, the 2,4-DSPBN or pharmaceutically acceptable salt thereof is administered to the subject orally, intravenously, subcutaneously, sublingually, subdermally, intrathecally, by inhalation, or locally within an ear.

In one embodiment, the method further comprises administering one or more compounds selected from the group consisting of N-acetylcysteine, Acetyl-L-Carnitine, glutathione monoethylester, ebselen, D-methionine, carbamathione and Szeto-Schiller peptides and their functional analogs. In another embodiment, the method further comprises administering N-acetylcysteine.

In one embodiment, the accumulated pathologic Tau protein is caused by aging. In one embodiment, the 2,4-DSPBN or pharmaceutically acceptable salt thereof is administered to a subject who is at least 60 years old, or at least 65 years old, or at least 70 years old, or at least 75 years old, or at least 80 years old.

In one embodiment, the accumulated pathologic Tau protein is caused by a central nervous system disease. In one embodiment, the 2,4-DSPBN or pharmaceutically acceptable salt thereof is administered to the subject at least two months, at least three months, at least six months, at least nine months, or at least twelve months after onset or diagnosis of the central nervous system disease. In another embodiment, the 2,4-DSPBN or pharmaceutically acceptable salt thereof is administered to the subject 1-60 months, 1-48 months, 1-24 months, 1-12 months, 3-60 months, 3-48 months, 3-24 months, 3-12 months, 6-60 months, 6-48 months, 6-24 months, 6-12 months, 9-60 months, 9-48 months, 9-24 months, 9-12 months, 12-60 months, 12-48 months, or 12-24 months after onset or diagnosis of the central nervous system disease.

In one embodiment, the accumulated pathologic Tau protein is caused by exposure to blast. In one embodiment, the 2,4-DSPBN or pharmaceutically acceptable salt thereof is administered to the subject at least two months, at least three months, at least six months, at least nine months, or at least twelve months after the exposure to blast. In another embodiment, the 2,4-DSPBN or pharmaceutically acceptable salt thereof is administered to the subject 1-60 months, 1-48 months, 1-24 months, 1-12 months, 3-60 months, 3-48 months, 3-24 months, 3-12 months, 6-60 months, 6-48 months, 6-24 months, 6-12 months, 9-60 months, 9-48 months, 9-24 months, 9-12 months, 12-60 months, 12-48 months, or 12-24 months after the exposure to blast.

In one embodiment, the accumulated pathologic Tau protein is caused by exposure to noise. In one embodiment, the 2,4-DSPBN or pharmaceutically acceptable salt thereof is administered to the subject at least two months, at least three months, at least six months, at least nine months, or at least twelve months after the exposure to noise. In another embodiment, the 2,4-DSPBN or pharmaceutically acceptable salt thereof is administered to the subject 1-60 months, 1-48 months, 1-24 months, 1-12 months, 3-60 months, 3-48 months, 3-24 months, 3-12 months, 6-60 months, 6-48 months, 6-24 months, 6-12 months, 9-60 months, 9-48 months, 9-24 months, 9-12 months, 12-60 months, 12-48 months, or 12-24 months after the exposure to noise.

In one embodiment, the accumulated pathologic Tau proteins is caused by infection. In one embodiment, the 2,4-DSPBN or pharmaceutically acceptable salt thereof is administered to the subject at least two months, at least three months, at least six months, at least nine months, or at least twelve months after the infection. In another embodiment, the 2,4-DSPBN or pharmaceutically acceptable salt thereof is administered to the subject 1-60 months, 1-48 months, 1-24 months, 1-12 months, 3-60 months, 3-48 months, 3-24 months, 3-12 months, 6-60 months, 6-48 months, 6-24 months, 6-12 months, 9-60 months, 9-48 months, 9-24 months, 9-12 months, 12-60 months, 12-48 months, or 12-24 months after the infection.

In one embodiment, the accumulated pathologic Tau protein is caused by exposure to toxin. In one embodiment, the 2,4-DSPBN or pharmaceutically acceptable salt thereof is administered to the subject at least two months, at least three months, at least six months, at least nine months, or at least twelve months the exposure to toxin. In another embodiment, the 2,4-DSPBN or pharmaceutically acceptable salt thereof is administered to the subject 1-60 months, 1-48 months, 1-24 months, 1-12 months, 3-60 months, 3-48 months, 3-24 months, 3-12 months, 6-60 months, 6-48 months, 6-24 months, 6-12 months, 9-60 months, 9-48 months, 9-24 months, 9-12 months, 12-60 months, 12-48 months, or 12-24 months after the exposure to toxin.

In one embodiment, the subject suffers from a central nervous system disease, and the administration of the 2,4-DSPBN or pharmaceutically acceptable salt thereof reduces the accumulated pathologic Tau protein in the central nervous system by at least 1%, or at least 2%, or at least 5%, or at least 10%, or at least 20%, or at least 50%.

In one embodiment, the subject suffers from chronic noise-induced or blast-induced hearing loss or tinnitus, or Presbycusis or Presbycusis-associated tinnitus, and the administration of the 2,4-DSPBN or pharmaceutically acceptable salt thereof reduces the accumulated pathologic Tau protein in the auditory system (e.g., in the cochlear or vestibular region) by at least 1%, or at least 2%, or at least 5%, or at least 10%, or at least 20%, or at least 50%.

In some embodiments, the method further comprises administering a Tau aggregation inhibitor. The Tau aggregation inhibitor can be a covalent or non-covalent inhibitor. Non-limiting examples of Tau aggregation inhibitors include curcumin, molecular tweezers (e.g., CLR01), phthalocyanine tetrasulfonate, oleocanthal, cinnamaldehyde, baicalein, isoprenaline, dopamine, dobutamine, levodopa, levodopa/carbidopa, trimetoquinol, hexoprenaline, methyldopa, and droxidopa.

Methods for Treating Central Nervous System Diseases

The invention described herein also provides a method for reducing accumulated pathologic Tau protein in the central nervous system of a subject in need thereof, comprising administering to the subject an effective amount of 2,4-DSPBN or a pharmaceutically acceptable salt thereof, wherein the subject suffers from a central nervous system disease or condition selected from chronic traumatic encephalopathy, Alzheimer's disease, Parkinson's disease, progressive supranuclear palsy, frontotemporal dementia, Pick's disease, Argyrophilic grain dementia, corticobasal degeneration, progressive subcortical gliosis, amyotrophic lateral sclerosis, diffuse neurofibrillary tangles with calcification, dementia pugilistica, tangle-only dementia, Down's syndrome, Gerstmann-Straussler-Scheinker disease, Hallervorden-Spatz disease, Creutzfeldt-Jakob disease, multiple system atrophy, Niemann-Pick disease type C, prion protein cerebral amyloid angiopathy, subacute sclerosing panencephalitis, myotonic dystrophy, non-Guanamian motor neuron disease with neurofibrillary tangles, and postencephalitic parkinsonism. Optionally, the 2,4-DSPBN is co-administered with an effective amount of NAC. The 2,4-DSPBN and NAC can be co-administered simultaneously or sequentially. The 2,4-DSPBN and NAC can be co-administered in one composition or in a separately compositions.

A further aspect of the invention relates to a method for reducing accumulated pathologic Tau protein in a patient suffering from chronic traumatic encephalopathy (CTE). In some embodiments, cerebrospinal fluid of the patient contains at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein. In some embodiments, an effective amount of 2,4-DSPBN or its pharmaceutically acceptable salt and optionally NAC are administered to a human patient suffering from CTE, wherein the 2,4-DSPBN or its pharmaceutically acceptable salt and the optional NAC reduce the amount of accumulated pathologic Tau proteins in the central nervous system to a level sufficient to deliver a therapeutic benefit to the patient against CTE.

A further aspect of the invention relates to a method for reducing accumulated pathologic Tau protein in a patient suffering from closed head trauma. In some embodiments, cerebrospinal fluid of the patient contains at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein. In some embodiments, an effective amount of 2,4-DSPBN or its pharmaceutically acceptable salt and optionally NAC are administered to a human patient suffering from closed head trauma, wherein the 2,4-DSPBN or its pharmaceutically acceptable salt and the optional NAC reduce the amount of accumulated pathologic Tau proteins in the central nervous system to a level sufficient to deliver a therapeutic benefit to the patient against closed head trauma.

A further aspect of the invention relates to a method for reducing accumulated pathologic Tau protein in a patient suffering from Alzheimer' s disease. In some embodiments, cerebrospinal fluid of the patient contains at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein. In some embodiments, an effective amount of 2,4-DSPBN or its pharmaceutically acceptable salt and optionally NAC are administered to a human patient suffering from Alzheimer's disease, wherein the 2,4-DSPBN or its pharmaceutically acceptable salt and the optional NAC reduce the amount of accumulated pathologic Tau proteins in the central nervous system to a level sufficient to deliver a therapeutic benefit to the patient against Alzheimer's disease.

A further aspect of the invention relates to a method for reducing accumulated pathologic Tau protein in a patient suffering from Parkinson's disease. In some embodiments, cerebrospinal fluid of the patient contains at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein. In some embodiments, an effective amount of 2,4-DSPBN or its pharmaceutically acceptable salt and optionally NAC are administered to a human patient suffering from Parkinson's disease, wherein the 2,4-DSPBN or its pharmaceutically acceptable salt and the optional NAC reduce the amount of accumulated pathologic Tau proteins in the central nervous system to a level sufficient to deliver a therapeutic benefit to the patient against Parkinson's disease.

A further aspect of the invention relates to a method for reducing accumulated pathologic Tau protein in a patient suffering from progressive supranuclear palsy (PSP). In some embodiments, cerebrospinal fluid of the patient contains at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein. In some embodiments, an effective amount of 2,4-DSPBN or its pharmaceutically acceptable salt and optionally NAC are administered to a human patient suffering from PSP, wherein the 2,4-DSPBN or its pharmaceutically acceptable salt and the optional NAC reduce the amount of accumulated pathologic Tau proteins in the central nervous system to a level sufficient to deliver a therapeutic benefit to the patient against PSP.

A further aspect of the invention relates to a method for reducing accumulated pathologic Tau protein in a patient suffering from frontotemporal dementia (FTD). In some embodiments, cerebrospinal fluid of the patient contains at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein. In some embodiments, an effective amount of 2,4-DSPBN or its pharmaceutically acceptable salt and optionally NAC are administered to a human patient suffering from FTD, wherein the 2,4-DSPBN or its pharmaceutically acceptable salt and the optional NAC reduce the amount of accumulated pathologic Tau proteins in the central nervous system to a level sufficient to deliver a therapeutic benefit to the patient against FTD.

A further aspect of the invention relates to a method for reducing accumulated pathologic Tau protein in a patient suffering from Pick's disease. In some embodiments, cerebrospinal fluid of the patient contains at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein. In some embodiments, an effective amount of 2,4-DSPBN or its pharmaceutically acceptable salt and optionally NAC are administered to a human patient suffering from Pick's disease, wherein the 2,4-DSPBN or its pharmaceutically acceptable salt and the optional NAC reduce the amount of accumulated pathologic Tau proteins in the central nervous system to a level sufficient to deliver a therapeutic benefit to the patient against Pick's disease.

A further aspect of the invention relates to a method for reducing accumulated pathologic Tau protein in a patient suffering from Argyrophilic grain dementia (AGD). In some embodiments, cerebrospinal fluid of the patient contains at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein. In some embodiments, an effective amount of 2,4-DSPBN or its pharmaceutically acceptable salt and optionally NAC are administered to a human patient suffering from AGD, wherein the 2,4-DSPBN or its pharmaceutically acceptable salt and the optional NAC reduce the amount of accumulated pathologic Tau proteins in the central nervous system to a level sufficient to deliver a therapeutic benefit to the patient against AGD.

A further aspect of the invention relates to a method for reducing accumulated pathologic Tau protein in a patient suffering from corticobasal degeneration (CBD). In some embodiments, cerebrospinal fluid of the patient contains at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein. In some embodiments, an effective amount of 2,4-DSPBN or its pharmaceutically acceptable salt and optionally NAC are administered to a human patient suffering from CBD, wherein the 2,4-DSPBN or its pharmaceutically acceptable salt and the optional NAC reduce the amount of accumulated pathologic Tau proteins in the central nervous system to a level sufficient to deliver a therapeutic benefit to the patient against CBD.

A further aspect of the invention relates to a method for reducing accumulated pathologic Tau protein in a patient suffering from progressive subcortical gliosis (PSG). In some embodiments, cerebrospinal fluid of the patient contains at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein. In some embodiments, an effective amount of 2,4-DSPBN or its pharmaceutically acceptable salt and optionally NAC are administered to a human patient suffering from PSG, wherein the 2,4-DSPBN or its pharmaceutically acceptable salt and the optional NAC reduce the amount of accumulated pathologic Tau proteins in the central nervous system to a level sufficient to deliver a therapeutic benefit to the patient against PSG.

A further aspect of the invention relates to a method for reducing accumulated pathologic Tau protein in a patient suffering from amyotrophic lateral sclerosis (ALS). In some embodiments, cerebrospinal fluid of the patient contains at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein. In some embodiments, an effective amount of 2,4-DSPBN or its pharmaceutically acceptable salt and optionally NAC are administered to a human patient suffering from ALS, wherein the 2,4-DSPBN or its pharmaceutically acceptable salt and the optional NAC reduce the amount of accumulated pathologic Tau proteins in the central nervous system to a level sufficient to deliver a therapeutic benefit to the patient against ALS.

A further aspect of the invention relates to a method for reducing accumulated pathologic Tau protein in a patient suffering from diffuse neurofibrillary tangles with calcification (DNTC). In some embodiments, cerebrospinal fluid of the patient contains at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein. In some embodiments, an effective amount of 2,4-DSPBN or its pharmaceutically acceptable salt and optionally NAC are administered to a human patient suffering from DNTC, wherein the 2,4-DSPBN or its pharmaceutically acceptable salt and the optional NAC reduce the amount of accumulated pathologic Tau proteins in the central nervous system to a level sufficient to deliver a therapeutic benefit to the patient against DNTC.

A further aspect of the invention relates to a method for reducing accumulated pathologic Tau protein in a patient suffering from dementia pugilistica (DP). In some embodiments, cerebrospinal fluid of the patient contains at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein. In some embodiments, an effective amount of 2,4-DSPBN or its pharmaceutically acceptable salt and optionally NAC are administered to a human patient suffering from DP, wherein the 2,4-DSPBN or its pharmaceutically acceptable salt and the optional NAC reduce the amount of accumulated pathologic Tau proteins in the central nervous system to a level sufficient to deliver a therapeutic benefit to the patient against DP.

A further aspect of the invention relates to a method for reducing accumulated pathologic Tau protein in a patient suffering from tangle-only dementia (TOD). In some embodiments, cerebrospinal fluid of the patient contains at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein. In some embodiments, an effective amount of 2,4-DSPBN or its pharmaceutically acceptable salt and optionally NAC are administered to a human patient suffering from TOD, wherein the 2,4-DSPBN or its pharmaceutically acceptable salt and the optional NAC reduce the amount of accumulated pathologic Tau proteins in the central nervous system to a level sufficient to deliver a therapeutic benefit to the patient against TOD.

A further aspect of the invention relates to a method for reducing accumulated pathologic Tau protein in a patient suffering from Down's syndrome. In some embodiments, cerebrospinal fluid of the patient contains at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein. In some embodiments, an effective amount of 2,4-DSPBN or its pharmaceutically acceptable salt and optionally NAC are administered to a human patient suffering from Down's syndrome, wherein the 2,4-DSPBN or its pharmaceutically acceptable salt and the optional NAC reduce the amount of accumulated pathologic Tau proteins in the central nervous system to a level sufficient to deliver a therapeutic benefit to the patient against Down's syndrome.

A further aspect of the invention relates to a method for reducing accumulated pathologic Tau protein in a patient suffering from Gerstmann-Straussler-Scheinker disease (GSS). In some embodiments, cerebrospinal fluid of the patient contains at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein. In some embodiments, an effective amount of 2,4-DSPBN or its pharmaceutically acceptable salt and optionally NAC are administered to a human patient suffering from GSS, wherein the 2,4-DSPBN or its pharmaceutically acceptable salt and the optional NAC reduce the amount of accumulated pathologic Tau proteins in the central nervous system to a level sufficient to deliver a therapeutic benefit to the patient against GSS.

A further aspect of the invention relates to a method for reducing accumulated pathologic Tau protein in a patient suffering from Hallervorden-Spatz disease (HSD). In some embodiments, cerebrospinal fluid of the patient contains at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein. In some embodiments, an effective amount of 2,4-DSPBN or its pharmaceutically acceptable salt and optionally NAC are administered to a human patient suffering from HSD, wherein the 2,4-DSPBN or its pharmaceutically acceptable salt and the optional NAC reduce the amount of accumulated pathologic Tau proteins in the central nervous system to a level sufficient to deliver a therapeutic benefit to the patient against HSD.

A further aspect of the invention relates to a method for reducing accumulated pathologic Tau protein in a patient suffering from Creutzfeldt-Jakob disease (CJD). In some embodiments, cerebrospinal fluid of the patient contains at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein. In some embodiments, an effective amount of 2,4-DSPBN or its pharmaceutically acceptable salt and optionally NAC are administered to a human patient suffering from CJD, wherein the 2,4-DSPBN or its pharmaceutically acceptable salt and the optional NAC reduce the amount of accumulated pathologic Tau proteins in the central nervous system to a level sufficient to deliver a therapeutic benefit to the patient against CJD.

A further aspect of the invention relates to a method for reducing accumulated pathologic Tau protein in a patient suffering from multiple system atrophy (MSA). In some embodiments, cerebrospinal fluid of the patient contains at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein. In some embodiments, an effective amount of 2,4-DSPBN or its pharmaceutically acceptable salt and optionally NAC are administered to a human patient suffering from MSA, wherein the 2,4-DSPBN or its pharmaceutically acceptable salt and the optional NAC reduce the amount of accumulated pathologic Tau proteins in the central nervous system to a level sufficient to deliver a therapeutic benefit to the patient against MSA.

A further aspect of the invention relates to a method for reducing accumulated pathologic Tau protein in a patient suffering from Niemann-Pick disease type C (NPC). In some embodiments, cerebrospinal fluid of the patient contains at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein. In some embodiments, an effective amount of 2,4-DSPBN or its pharmaceutically acceptable salt and optionally NAC are administered to a human patient suffering from NPC, wherein the 2,4-DSPBN or its pharmaceutically acceptable salt and the optional NAC reduce the amount of accumulated pathologic Tau proteins in the central nervous system to a level sufficient to deliver a therapeutic benefit to the patient against NPC.

A further aspect of the invention relates to a method for reducing accumulated pathologic Tau protein in a patient suffering from prion protein cerebral amyloid angiopathy (PrP-CAA). In some embodiments, cerebrospinal fluid of the patient contains at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein. In some embodiments, an effective amount of 2,4-DSPBN or its pharmaceutically acceptable salt and optionally NAC are administered to a human patient suffering from PrP-CAA, wherein the 2,4-DSPBN or its pharmaceutically acceptable salt and the optional NAC reduce the amount of accumulated pathologic Tau proteins in the central nervous system to a level sufficient to deliver a therapeutic benefit to the patient against PrP-CAA.

A further aspect of the invention relates to a method for reducing accumulated pathologic Tau protein in a patient suffering from subacute sclerosing panencephalitis (SSPE). In some embodiments, cerebrospinal fluid of the patient contains at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein. In some embodiments, an effective amount of 2,4-DSPBN or its pharmaceutically acceptable salt and optionally NAC are administered to a human patient suffering from SSPE, wherein the 2,4-DSPBN or its pharmaceutically acceptable salt and the optional NAC reduce the amount of accumulated pathologic Tau proteins in the central nervous system to a level sufficient to deliver a therapeutic benefit to the patient against SSPE.

A further aspect of the invention relates to a method for reducing accumulated pathologic Tau protein in a patient suffering from myotonic dystrophy. In some embodiments, cerebrospinal fluid of the patient contains at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein. In some embodiments, an effective amount of 2,4-DSPBN or its pharmaceutically acceptable salt and optionally NAC are administered to a human patient suffering from myotonic dystrophy, wherein the 2,4-DSPBN or its pharmaceutically acceptable salt and the optional NAC reduce the amount of accumulated pathologic Tau proteins in the central nervous system to a level sufficient to deliver a therapeutic benefit to the patient against myotonic dystrophy.

A further aspect of the invention relates to a method for reducing accumulated pathologic Tau protein in a patient suffering from non-Guanamian motor neuron disease with neurofibrillary tangles. In some embodiments, cerebrospinal fluid of the patient contains at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein. In some embodiments, an effective amount of 2,4-DSPBN or its pharmaceutically acceptable salt and optionally NAC are administered to a human patient suffering from non-Guanamian motor neuron disease with neurofibrillary tangles, wherein the 2,4-DSPBN or its pharmaceutically acceptable salt and the optional NAC reduce the amount of accumulated pathologic Tau proteins in the central nervous system to a level sufficient to deliver a therapeutic benefit to the patient against non-Guanamian motor neuron disease with neurofibrillary tangles.

A further aspect of the invention relates to a method for reducing accumulated pathologic Tau protein in a patient suffering from postencephalitic parkinsonism (PEP). In some embodiments, cerebrospinal fluid of the patient contains at least 0.05 ng/ml, or at least 0.07 ng/ml, or at least 0.1 ng/ml, or at least 0.15 ng/ml, or at least 0.2 ng/ml, or at least 0.25 ng/ml, or at least 0.3 ng/ml, or at least 0.35 ng/ml, or at least 0.4 ng/ml, or at least 0.45 ng/ml, or at least 0.5 ng/ml, of accumulated pathologic Tau protein. In some embodiments, an effective amount of 2,4-DSPBN or its pharmaceutically acceptable salt and optionally NAC are administered to a human patient suffering from PEP, wherein the 2,4-DSPBN or its pharmaceutically acceptable salt and the optional NAC reduce the amount of accumulated pathologic Tau proteins in the central nervous system to a level sufficient to deliver a therapeutic benefit to the patient against PEP.

In some embodiments, by reversing accumulation of pathologic Tau protein, the present invention slows, stops or reverses neurodegeneration in the central nervous system of a patient suffering from a central nervous system disease or condition selected from chronic traumatic encephalopathy, Alzheimer's disease, Parkinson's disease, progressive supranuclear palsy, frontotemporal dementia, Pick's disease, Argyrophilic grain dementia, corticobasal degeneration, progressive subcortical gliosis, amyotrophic lateral sclerosis, diffuse neurofibrillary tangles with calcification, dementia pugilistica, tangle-only dementia, Down's syndrome, Gerstmann-Straussler-Scheinker disease, Hallervorden-Spatz disease, Creutzfeldt-Jakob disease, multiple system atrophy, Niemann-Pick disease type C, prion protein cerebral amyloid angiopathy, subacute sclerosing panencephalitis, myotonic dystrophy, non-Guanamian motor neuron disease with neurofibrillary tangles, and postencephalitic parkinsonism.

### WORKING EXAMPLES

The following examples are for illustrative purposes only and should not be interpreted as limitations of the claimed invention. There are a variety of alternative techniques and procedures available to those of skill in the art which would similarly permit one to successfully perform the intended invention.

### Example 1

### Reversal of Noise-Induced Accumulation of Pathologic T22 Tau in the Spiral Ganglion of the Cochlea

FIG. 1 shows T22-positive neuron counting and statistical analyses in the spiral ganglion (SG) after noise exposure. Rats were exposed to 115 dB SPL octave band noise for one hour. One group of rats were treated with 2,4-DSPBN and NAC (300 mg/kg for each) one hour after noise exposure and twice/day for the next two days (total 5 doses, the N/T group) while another group of rats were treated with vehicle (saline) only (the N group). One group of rats without noise exposure served as normal controls (the NC group). Animals were euthanized at 7- and 21-days after noise exposure, and cochlear tissues were processed for immunostaining. An increased number of T22-positive neurons was observed in the SG at 7 days after noise exposure in the 7D-N/T and 7D-N groups compared to the NC group (**, *** p<0.01 or 0.001). No treatment effect was observed at this time point after noise exposure (p>0.05). At twenty-one days after noise exposure, an increased number of T22-positive neurons was observed in the SG only in the vehicle-treated group (21D-N, *** p<0.001) but not in the group treated with a combination of 2,4-DSPBN+NAC (21D-N/T, p>0.05), indicating a treatment effect at this time point (p<0.05).

### Example 2

### 2,4-DSPBN+NAC Treatment Significantly Reduced the Accumulation of Insoluble Cytotoxic Tau Aggregates in the Brains of Tau(P301S) Mice

Transgenic (Tg) Tau(P301S) mice are an aggressive tauopathy model that begins demonstrating neurodegeneration and accumulation of hyperphosphorylated Tau oligomers within the first five months of age. Tau(P301S) mice were treated intraperitoneally (i.p.) daily for three months with either placebo (saline) or 2,4-DSPBN+NAC (300 mg/kg of each), beginning at an age of 2.5 months. Differential fractionation of brain tissues and immunoblot analyses were conducted among the Tg mice at 5.5 months to measure the relative accumulation of detergent (sarkosyl)-insoluble aggregates of hyperphosphorylated Tau protein in animals treated with the placebo or the active drug combination, using methodology described by Berger et al., J Neurosci. 27(14):3650-62 (2007). FIG. 2A depicts an example of immunoblot analyses conducted among the sarkosyl-insoluble fractions isolated from hippocampal (HCa) brain tissues from Tau(P301S)-Tg mice treated with either placebo or 2,4-DSPBN+NAC. In FIG. 2A, the relative amounts of aggregated hyperphosphorylated Tau (p-Tau (S202, T205)) that remained in the sarkosyl-insoluble pellet of the HCa extracts were detected, using an antibody (AT8) specific to p-Tau (S202,T205). The levels of aggregated insoluble p-Tau(S202, T205) in P301S-Tg mice treated with 2,4-DSPBN+NAC were noticeably reduced relative to placebo-treated controls. FIG. 2B is a graphical summary depicting quantitative comparisons of relative densitometric immunoreactivity of p-Tau in the sarkosyl-insoluble fraction of the HCa from mice in each test group (n=6 mice/group). These data showed a statistically-significant reduction in the amount of cytotoxic sarkosyl-insoluble p-Tau aggregation in Tg mice treated with 2,4-DSPBN+NAC. Immunoblots of the housekeeping protein, GAPDH, in the soluble supernatant fractions from the same samples served as internal loading controls for quantitative comparisons.

### EXAMPLE 3

### 2,4-DSPBN+NAC Treatment Reduced Okadaic Acid-Induced p-Tau Accumulation in SH-SY5Y Neuroblastoma Cells

Reproducible hyperphosphorylated p-Tau accumulation can be induced in the SH-SY5Y neuroblastoma cell line following incubation with Okadaic Acid (OA), a small molecule inhibitor of protein phosphatase 2A (PP2A), according to the method described by Boban et al., J Neurosci Methods., pii: S0165-0270(18)30297-8 (2018). In this experiment, SH-SY5Y cells were incubated with 100 nM OA alone or in combination with 2,4-DSPBN+NAC for three hours prior to harvesting the cells for immunoblot evaluations with the AT8 phospho-Tau (S202,T205) antibody or the housekeeping protein, GAPDH, as shown in FIG. 3. In cells treated with OA alone, marked phospho-Tau(S202,T205) accumulation was observed relative to untreated control cells (compare lanes 1 and 2). Cells treated with OA and 2,4-DSPBN+NAC, showed markedly reduced accumulation of p-Tau levels relative to cells treated with OA alone (compare lanes 2 and 4 or lanes 2 and 5). In the absence of OA, 2,4-DSPBN+NAC treatment (500 μM NAC, 185 μM 2,4-DSPBN) alone also seemingly reduced the homeostatic baseline levels of p-Tau in SH-SY5Y cells (compare lanes 1 and 3). Two doses of combinatorial 2,4-DSPBN+NAC (185 μM 2,4-DSPBN/500 μM NAC or 93 μM 2,4-DSPBN/250 μM NAC) were used in this experiment, each of which used a molar ratio that mimics the equal mass:mass ratios used in the in vivo studies (i.e. a 1:1 mass ratio of 2,4-DSPBN and NAC is equivalent to a 1:2.7 molar ratio).

Quantitative evaluations demonstrated a statistically-significant reduction in okadaic acid-induced p-Tau accumulation in SH-SY5Y neuroblastoma cells treated with 2,4-DSPBN+NAC, as shown in FIG. 4. Biological and technical replicates of the experiment described in FIG. 3 were conducted, and p-Tau(S202, T205) immunoblots (AT8 antibody) were subjected to densitometric evaluations of relative protein levels, using NIH ImageJ software and the internal GAPDH protein standard. Based on these evaluations, it was demonstrated that 2,4-DSPBN+NAC treatment (500 μM NAC, 185 μM 2,4-DSPBN) promoted a statistically-significant reduction in OA-induced p-Tau accumulation, to a level that was statistically-indistinguishable from the levels observed in untreated control cells, indicative of a strong treatment effect.

### Example 4

### Okadaic Acid-Induced p-Tau Accumulation in SH-SY5Y Neuroblastoma Cells was Reduced by 2,4-DSPBN+NAC Treatment

FIG. 5 shows that 2,4-DSPBN+NAC treatment enhanced the reversal of accumulated phosphorylated Tau protein levels. In the pilot experiment depicted in FIG. 5, SH-SY5Y cells were challenged with OA alone for 3 hours and then chased with fresh media without OA but containing either saline or 2,4-DSPBN+NAC (500 μM NAC, 185 μM 2,4-DSPBN). Cells were then harvested at 30 min. post-treatment to examine whether 2,4-DSPBN+NAC treatment might accelerate the resolution of the accumulated p-Tau induced by OA. FIG. 5A depicts an immunoblot evaluation of hyperphosphorylated p-Tau(S202,T205) levels in SH-SY5Y cells. This data demonstrated a persistent, marked OA-induced accumulation that was still evident after 30 min in OA-free media, whereas cells treated with 2,4-DSPBN+NAC showed a detectable reduction at this same timepoint post-OA exposure. FIG. 5B shows quantitative densitometric evaluations of technical replicates of this experiment, which demonstrated that treatment with 2,4-DSPBN+NAC seemed to more rapidly resolve the OA-induced accumulation of p-Tau in SH-SY5Y cells.

Definitions

As used herein, the singular terms “a,” “an,” and “the” include plural referents unless the context clearly dictates otherwise. Thus, for example, reference to a compound can include multiple compounds unless the context clearly dictates otherwise.

As used herein, the terms “substantially,” “substantial,” and “about” are used to describe and account for small variations. When used in conjunction with an event or circumstance, the terms can refer to instances in which the event or circumstance occurs precisely as well as instances in which the event or circumstance occurs to a close approximation. For example, the terms can refer to less than or equal to ±10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations, which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scopes of this invention.

Equivalents

It is to be understood that while the disclosure has been described in conjunction with the above embodiments, the foregoing description and examples are intended to illustrate and not limit the scope of the disclosure. Other aspects, advantages and modifications within the scope of the disclosure will be apparent to those skilled in the art to which the disclosure pertains.

Unless otherwise defined, all technical and scientific terms used herein have the same meaning as commonly understood by one of ordinary skill in the art to which this disclosure belongs.

The embodiments illustratively described herein may suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by specific embodiments and optional features, modification, improvement, and variation of the embodiments therein herein disclosed may be resorted to by those skilled in the art, and that such modifications, improvements, and variations are considered to be within the scope of this disclosure. The materials, methods, and examples provided here are representative of particular embodiments, are exemplary, and are not intended as limitations on the scope of the disclosure.

The scope of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the disclosure are described in terms of Markush groups, those skilled in the art will recognize that embodiments of the disclosure may also thereby be described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

What is claimed is:

1. A method for reducing accumulated pathologic Tau proteins in a subject, comprising administering to said subject in need thereof a pharmaceutically effective amount of a composition comprising 2,4-disulfonyl α-phenyl tertiary butyl nitrone (2,4-DSPBN) or a pharmaceutically acceptable salt thereof and N-acetylcysteine (NAC) or a pharmaceutically acceptable salt thereof.

2. The method of claim 1, wherein the composition is administered as a pharmaceutical composition, which further comprises a pharmaceutically acceptable carrier.

3. The method of claim 1, wherein the composition is administered to the subject orally, intravenously, subcutaneously, sublingually, subdermally, intrathecally, by inhalation, or locally within an ear.

4. The method of claim 1, wherein the accumulated pathologic Tau protein is caused by aging.

5. The method of claim 1, wherein the accumulated pathologic Tau protein is caused by a central nervous system disease.

6. The method of claim 5, wherein the composition is administered to the subject at least two months, at least three months, at least six months, at least nine months, at least twelve months, or 1-60 months after onset of the central nervous system disease.

7. The method of claim 1, wherein the accumulated pathologic Tau protein is caused by exposure to noise.

8. The method of claim 7, wherein the composition is administered to the subject at least two months, at least three months, at least six months, at least nine months, at least twelve months, or 1-60 months after the exposure to noise.

9. The method of claim 1, wherein the accumulated pathologic Tau is caused by exposure to blast.

10. The method of claim 9, wherein the composition is administered to the subject at least two months, at least three months, at least six months, at least nine months, at least twelve months, or 1-60 months after the exposure to blast.

11. The method of claim 1, wherein the accumulated pathologic Tau is caused by infection.

12. The method of claim 11, wherein the composition is administered to the subject at least two months, at least three months, at least six months, at least nine months, at least twelve months, or 1-60 months after the infection.

13. The method of claim 1, wherein the accumulated pathologic Tau is caused by exposure to toxin.

14. The method of claim 13, wherein the composition is administered to the subject at least two months, at least three months, at least six months, at least nine months, at least twelve months, or 1-60 months after the exposure to toxin.

15. The method of claim 1, wherein cerebrospinal fluid of the subject comprises at least 0.07 ng/ml of accumulated pathologic Tau protein.

16. The method of claim 1, wherein the subject suffers from a central nervous system disease, wherein the administration of the composition reduces the accumulated pathologic Tau protein in the central nervous system by at least 5%.

17. The method of claim 1, wherein the subject suffers from chronic noise-induced or blast-induced hearing loss or tinnitus, or Presbycusis or Presbycusis-associated tinnitus, wherein the administration of the composition reduces the accumulated pathologic Tau protein in the cochlear or vestibular region by at least 5%.

18. The method of claim 1, further comprising administering a Tau aggregation inhibitor.

19. The method of claim 1, wherein the accumulated pathologic Tau proteins are hyperphosphorylated.

20. A method for reducing accumulated pathologic Tau proteins in a subject, comprising administering to said subject in need thereof a pharmaceutically effective amount of a composition comprising 2,4-disulfonyl α-phenyl tertiary butyl nitrone (2,4-DSPBN) or a pharmaceutically acceptable salt thereof.

21. The method of claim 20, wherein the accumulated pathologic Tau is caused by exposure to blast or noise, and wherein the composition is administered to the subject at least two months, at least three months, at least six months, at least nine months, at least twelve months, or 1-60 months after the exposure to blast or noise.

22. The method of claim 20, wherein the accumulated pathologic Tau proteins are hyperphosphorylated.

23. A composition for use in a method for reducing accumulated pathologic Tau proteins in a subject, comprising a pharmaceutically effective amount of a composition comprising 2,4-disulfonyl α-phenyl tertiary butyl nitrone (2,4-DSPBN) or a pharmaceutically acceptable salt thereof and N-acetylcysteine (NAC) or a pharmaceutically acceptable salt thereof.

24. A composition for use in a method for reducing accumulated pathologic Tau proteins in a subject, comprising a pharmaceutically effective amount of a composition comprising 2,4-disulfonyl α-phenyl tertiary butyl nitrone (2,4-DSPBN) or a pharmaceutically acceptable salt thereof.
